(19) United States
(12) Patent Application Publication
(10) Pub. No.: US 2006/0205041 A1
(43) Pub. Date: Sep. 14, 2006

(54) Recombinant bovine thrombin

(76) Inventors: Christopher Carl Frye, Plainfield, IN (US); Charles Lee Hershberger, Greenfield, IN (US)

Correspondence Address: ELI LILLY & COMPANY, PATENT DIVISION, P.O. BOX 6288, INDIANAPOLIS, IN, 46206-6288, US

(21) Appl. No.: 10/495,479

(22) PCT Filed: Dec. 5, 2002
(86) PCT No.: PCT/US02/36146

§ 371 Date: May 13, 2004

Related U.S. Application Data
(60) Provisional application No. 60/340,134, filed on Dec. 14, 2001.

(51) Int. Cl. C12P 21/06; C07H 21/04; C12N 9/74

(52) U.S. Cl. 435/100; 435/69.1; 435/214; 435/325; 435/320.1; 536/23.2

(57) ABSTRACT

Methods are disclosed for producing recombinant thrombin from precursor molecules that do not require activation by Factor Xa. The protein is produced from host cells transformed or transfected with DNA construct(s) containing information necessary to direct the expression of thrombin precursors. Methods for purification of the thrombin precursors are also described. Thrombin precursors produced from transformed or transfected host cells are auto-activated or secreted in an activated form.

The present invention is directed generally towards methods for producing recombinant proteins, and more specifically, towards methods producing recombinant bovine thrombin from host cells as precursor molecules which can be activated without Factor Xa.

Thrombin is a specific serine protease that is a member of the typsin like family of enzymes. It plays a central role in hemostasis and regulates both the procoagulant and the anticoagulant pathways (Fenton, J. W., Ann. N.Y. Acad. Sci., 370: 468-495, 1981). As a factor in coagulation, thrombin catalyzes the formation of insoluble fibrin thereby initiating the blood-clotting cascade. In the case of anticoagulation, thrombin interacts with thrombomodulin on the surface of endothelial cells and this interaction results in the subsequent activation of protein C (Esmon et al., J. Biol. Chem., 257:859-864,1982). In addition to its regulatory roles in hemostasis, thrombin is known to interact with a wide variety of cell types. These interactions lead to a broad range of effects including platelet aggregation, growth promotion, neurite retraction, and chemotactic response (Bizios, et al., J. Cell Physiol, 128:485-490, 1986).

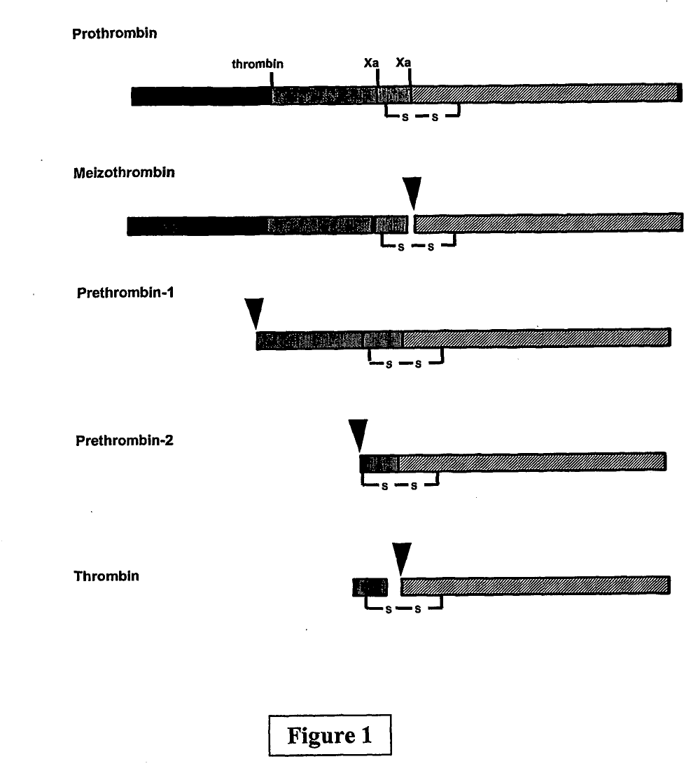

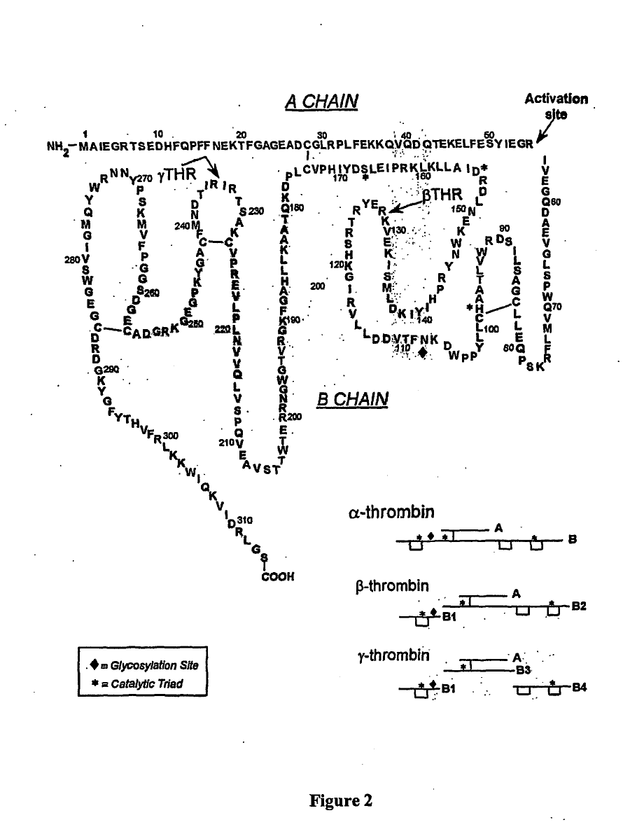

Thrombin is expressed and secreted as a zymogen and circulates as an inactive prothrombin precursor. The enzyme is activated to a functional state through a cascade that includes a key processing step involving the Factor Xa cleavage (FIG. 1). The final product (γ-thrombin) of the activation cascade is a 36-residue “A chain” linked covalently to a 256-residue “B chain” via a disulfide bridge, (FIG. 2). Thrombin is also known to possess significant post-translational modifications. The active form of the serum protein possesses a single N-linked glycosylation site, however the prothrombin form contains an additional glycosylation moiety, as well as, ten sites for y-carboxylation.

In addition to its role as a key physiological regulator, thrombin has become an enzyme of choice for the biotechnology industry. This protease is used in numerous commercially available microbial expression systems for cleavage of fusion peptides, as well as, for removal of purification and assay “tags”. The attractiveness of this protease lies in its catalytic efficiency and cleavage site specificity. This is somewhat surprising since thrombin is related to trypsin, a serine protease, which is known to cleave a wide range of polypeptide substrates. Thrombin is much more specific than trypsin and other members of this serine protease family. The enzyme recognizes the four amino acid sequence LVPR and numerous other sites. In commercial systems the cleavage sequence LVPRGS is used most frequently.

The current source of thrombin used in commercial processes is derived from bovine plasma. Since thrombin is an enzyme of choice for the biotechnology industry, a more effective commercial manufacturing process is needed to meet this growing demand. Importantly, a recombinant form of thrombin is needed since regulatory concerns currently exist around the use of animal source materials (ASM's) in manufacturing processes because of the risk of transmissible spongiform encephalopathies (TSEs), and in particular, bovine spongiform encephalopathy (BSE) or so called “mad cow disease”.

Thus, a need exists for an economically produced recombinant thrombin to address the regulatory concerns that surround the use of animal-source materials (i.e. glandular bovine thrombin) in manufacturing processes and to meet the growing demands for this enzyme in the biotechnology industry. The present invention fulfills these needs by providing two thrombin precursor molecules, prothrombin and prethrombin-2, which, upon activation, are essentially identical to the animal-derived thrombin in terms of kinetic characteristics and enzymatic stability across a range of temperature and pH values, but do not require activation by the factor Xa protease. Unexpectedly, recombinant prothrombin can be purified and subsequently auto-activated in vitro, while the recombinant prethrombin-2 precursor is secreted and purified as the fully active enzyme.

The present invention provides an isolated nucleic acid encoding a recombinant bovine thrombin precursor molecule wherein said precursor molecule is an engineered form of prothrombin and said isolated nucleic acid is expressed in CHO cells (SEQ ID NO:5) or E. coli cells (SEQ ID NO:1).

The present invention further provides an isolated nucleic acid encoding a recombinant bovine thrombin precursor molecule wherein said precursor molecule is an engineered form of prethrombin-2 and said isolated nucleic acid is expressed in CHO cells (SEQ ID NO:7) or E. coli cells (SEQ ID NO:3).

The present invention further provides a polynucleotide encoding a polypeptide having the amino acid sequence as shown in SEQ ID NO:6, wherein said polypeptide is the recombinant bovine thrombin precursor molecule prothrombin and said polynucleotide is expressed in CHO cells.

The present invention further provides a polynucleotide encoding a polypeptide having the amino acid sequence as shown in SEQ ID NO:2, wherein said polypeptide is the recombinant bovine thrombin precursor molecule prothrombin and said polynucleotide is expressed in E. coli cells.

The present invention further provides a polynucleotide encoding a polypeptide having the amino acid sequence as shown in SEQ ID NO:8, wherein said polypeptide is the recombinant bovine thrombin precursor molecule prethrombin-2 and said polynucleotide is expressed in CHO cells.

The present invention further provides a polynucleotide encoding a polypeptide having the amino acid sequence as shown in SEQ ID NO:4, wherein said polypeptide is the recombinant bovine thrombin precursor molecule prethrombin-2 and said polynucleotide is expressed in E. coli cells.

The present invention further provides methods of purifying said bovine thrombin precursor molecules and methods of using the same.

FIG. 1. Activation scheme for prothrombin. Thrombin is a serum protein that is secreted (prepro form not shown here) as an inactive “pro form” precursor molecule (prothrombin). Through the action of pre-existing and freely-circulating thrombin, as well as Factor Xa, the prothrombin molecule is cleaved to generate fully functional and active thrombin, which consists of two peptide chains (A chain and B chain, respectively) that are linked via a single disulfide bond. Proteolytic cleavage sites are designated by ▾. Prethrombin-2 represents the smallest single chain precursor to thrombin.

FIG. 2. Recombinant thrombin structure with wild type (Factor Xa) activation sequences. Cartoon depicts the 2-chain protein. The native “A” chain consists of 36 amino acids (here shown as the “recombinant version” which has 55 residues). The “B” chain consists of 259 amino acid residues. Disulfide linkages, catalytic residues and glycolsylation site are marked as described in the key box. In addition, the generic structures of three thrombin species are shown in the lower right of this figure.

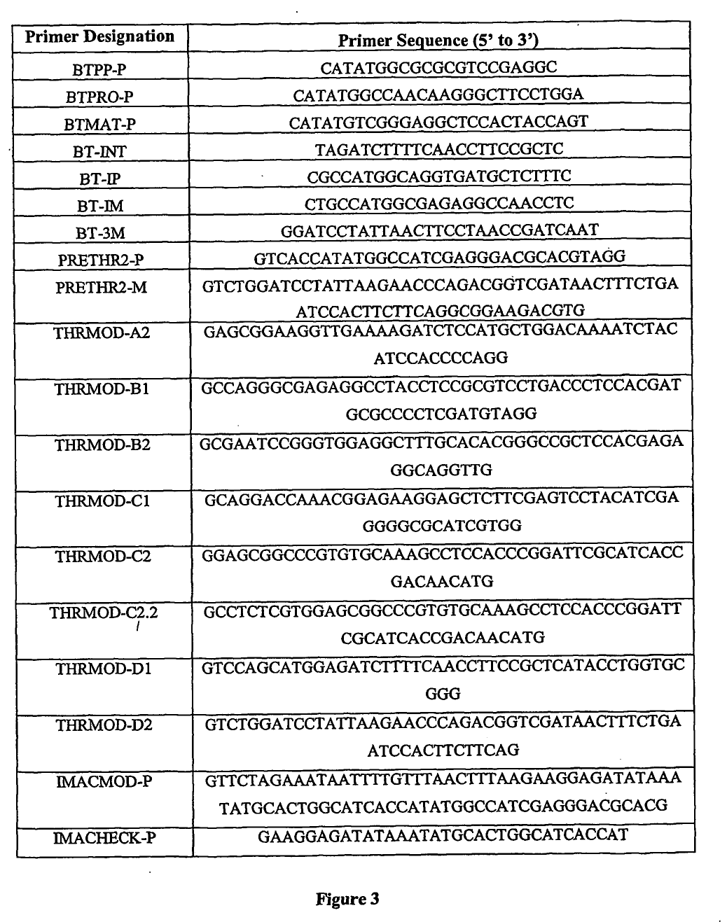

FIG. 3. Primers designed to amplify and modify the cDNA sequences coding for several precursors to thrombin.

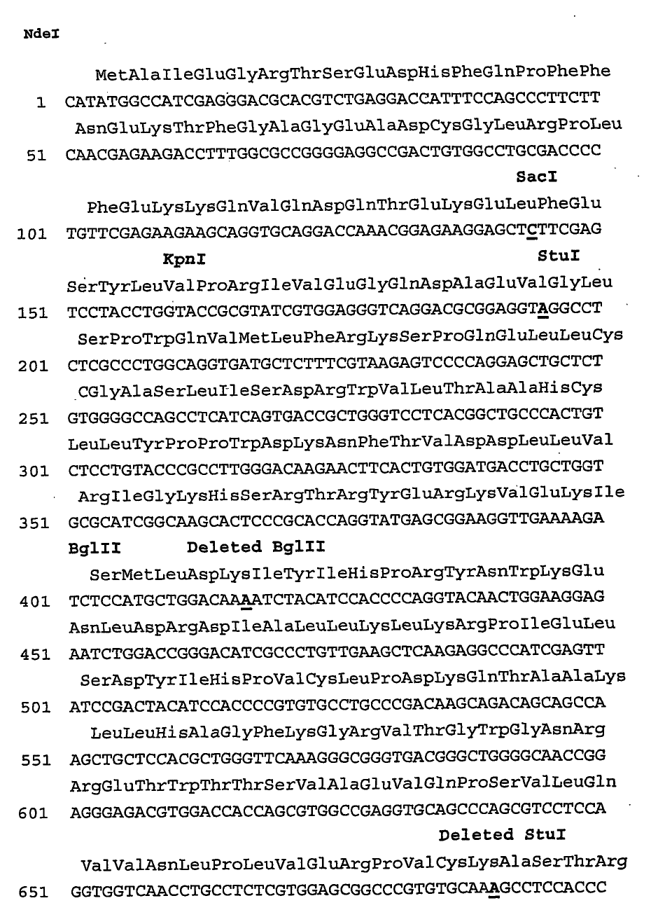

FIG. 4. Prethrombin-2 expression cassette modifications for expression in E. coli. The complete expression cassette (shown 5′ to 3′) was amplified as a NdeI to BamHI fragment. The internal modifications were introduced by overlap extension mutagensis (OEM) PCR. Changes are noted by bold type and underlining.

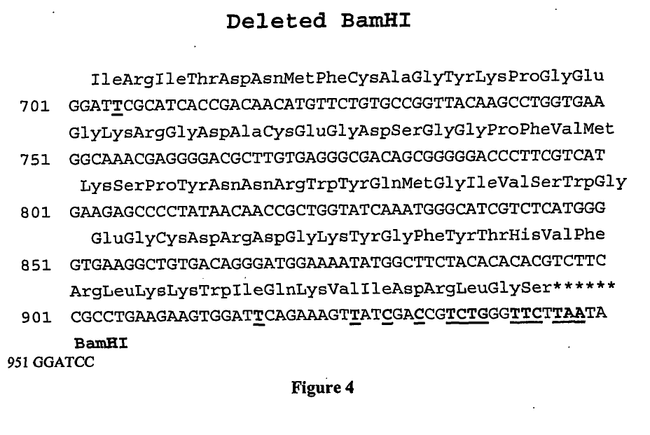

FIG. 5. Amino acid sequence derived from translation of the nucleotide sequence (for preprothrombin) obtained from Genbank database but containing amino acid changes determined following cloning of the cDNA. Note that there are three amino acid changes found in the gene cloned from bovine liver cDNA compared to Genbank database sequence. These changes are noted in bold in the figure. The 43 amino acid leader sequence (or signal peptide) is underlined. The “pro” portion or “activation” peptide (residues #'s 44-314) of the molecule is italicized. The putative N-linked glycosylation sites (asparagine residues) are at residues 144 and 419. The Factor Xa cleavage sites (natural activation) are underlined with wavy script. The asterisk indicates the stop codon. The specific amino acid changes observed are as follows: arginine 95 to lysine, histidine 230 to serine and histidine 248 to aspartic acid.

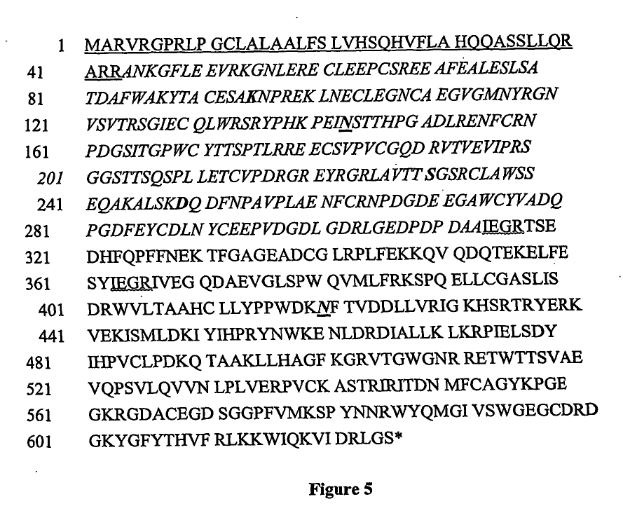

FIG. 6. Initial constructions for confirming cloned sequences from which the sequences of the present invention are derived.

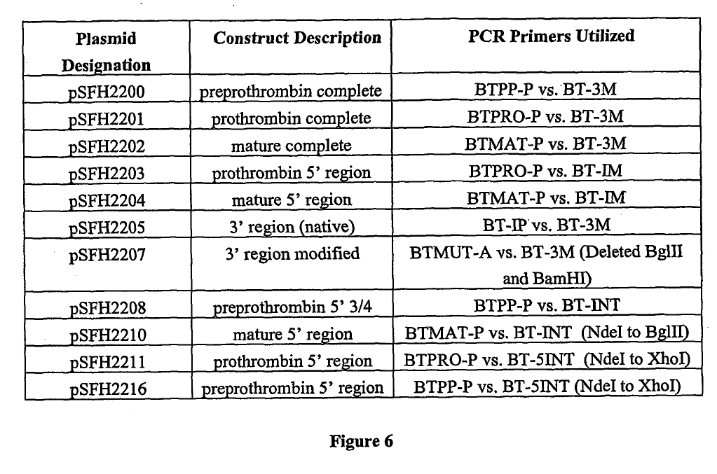

FIG. 7. Amino acid sequence of Factor Xa activated recombinant prethrombin-2 expression cassette. Note the Factor Xa cleavage sites are indicated by bold type.

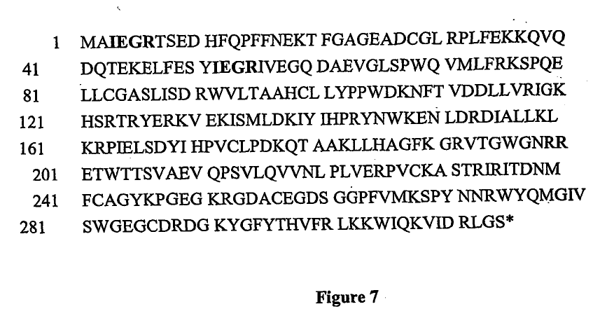

FIG. 8. Alternative activation DNA linker sequences. The nucleotide and resulting translation products for each oligonucleotide adaptor are shown. The adaptors were cloned directly into the pSFH2250 backbone as SacI to StuI fragments. Where possible, the alternative cleavage cassettes were distinguished by introduction of a unique restriction site for subsequent diagnostic purposes.

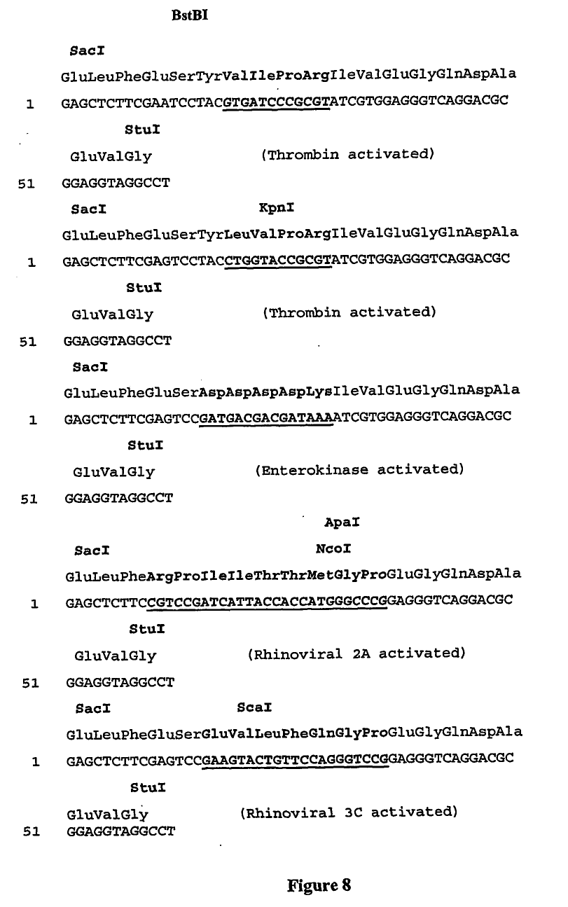

FIG. 9. Expression plasmids—E. coli alternative activation expression constructs.

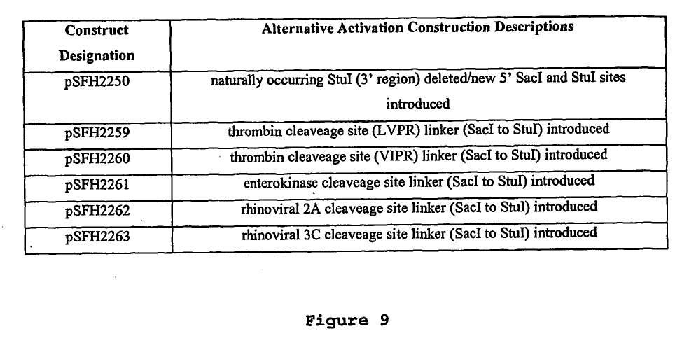

FIG. 10. Bacterial cloning and expression vector backbones.

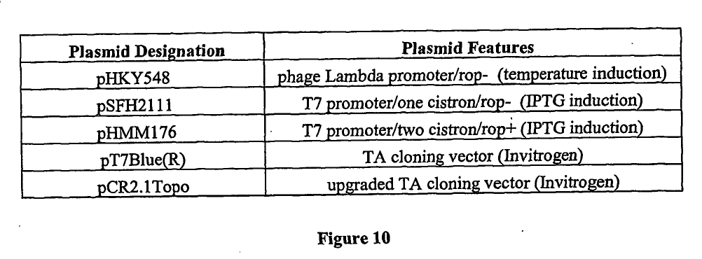

FIG. 11. IMAC handle sequence. “Tag” handle sequences for the N-terminal IMAC (NdeI) and C-terminal (BamHI) polyhis (6×) additions, respectively.

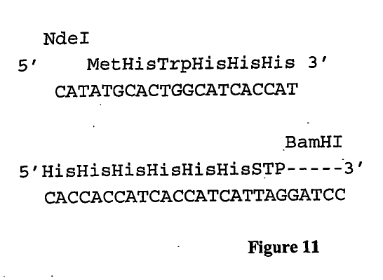

FIG. 12. Expression plasmids—CHO cell expression constructs.

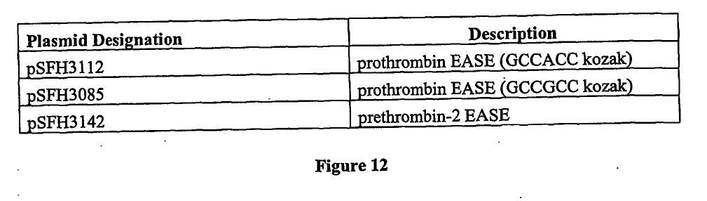

FIG. 13. Thrombin activated prothrombin nucleotide sequence, SEQ ID NO:1, expressed in E. coli.

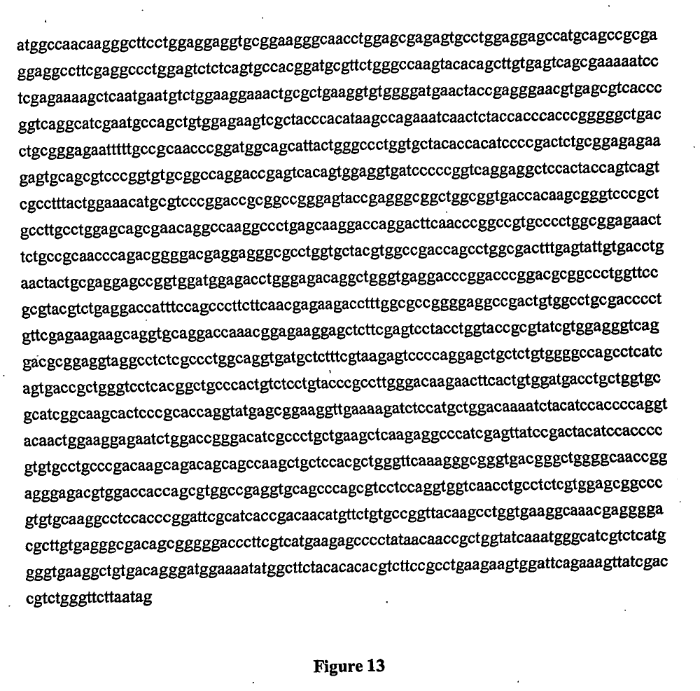

FIG. 14. Amino acid sequence, SEQ ID NO:6, of engineered prothrombin expressed in CHO cells (with C-terminal polyHis tag).

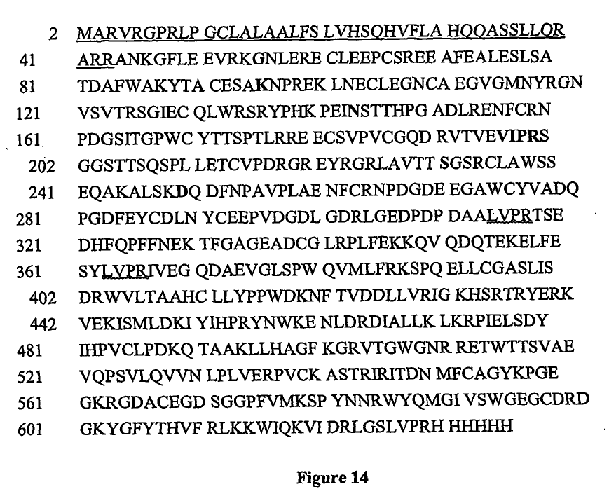

FIG. 15. Amino acid sequence, SEQ ID NO:2, of engineered prothrombin expressed in E. coli.

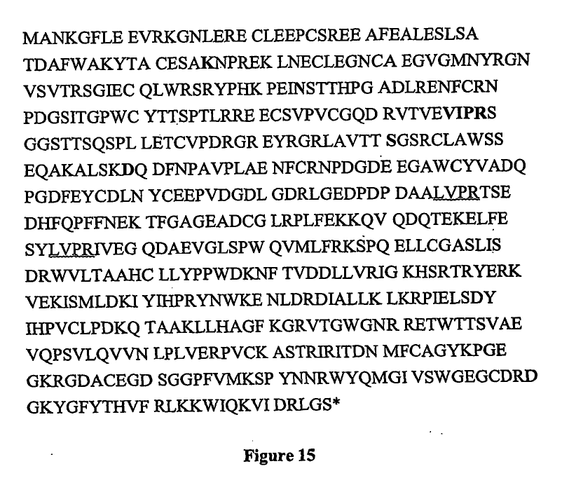

FIG. 16. Amino acid sequence, SEQ ID NO:4, of engineered prethrombin-2 expressed in E. coli.

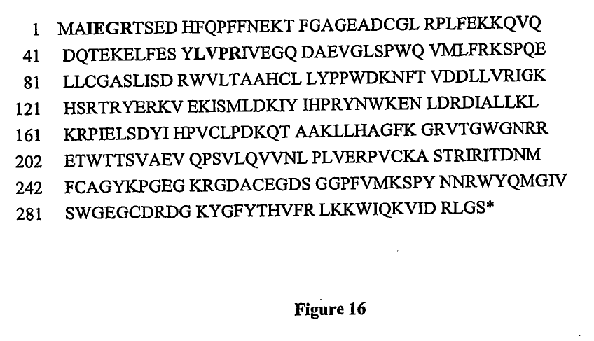

FIG. 17. Amino acid sequence, SEQ ID NO:8, of engineered prethrombin-2 expressed in CHO cells (with C-terminal polyHis tag).

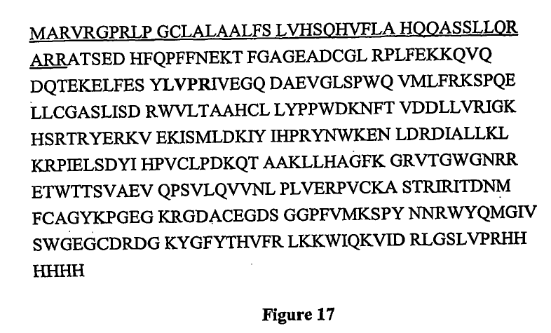

FIG. 18. E. coli engineered prethrombin-2 expression cassette (thrombin activated) nucleotide sequence, SEQ ID NO:3.

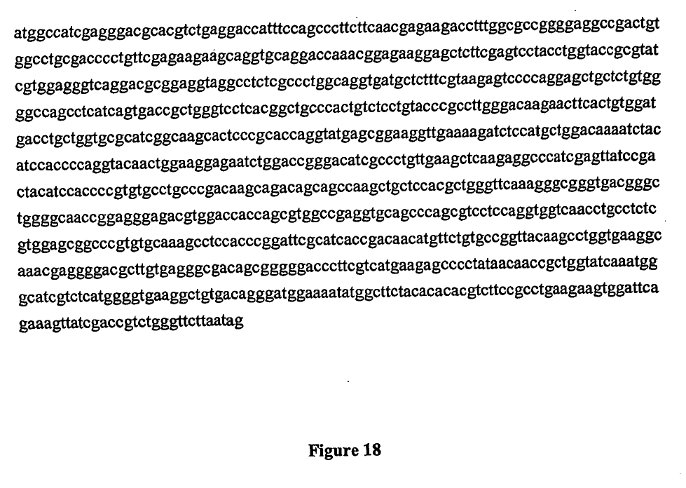

FIG. 19. Mammalian engineered prethrombin-2 including native preprothrombin leader sequence (thrombin activated with thrombin removable 3′ polyHis sequence tag) expression cassette nucleotide sequence, SEQ ID NO:7.

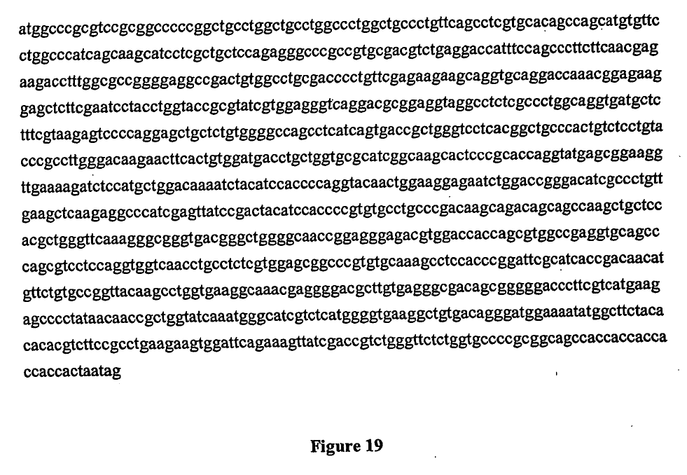

FIG. 20. Thrombin activatable prothrombin nucleotide sequence, SEQ ID NO:5, expressed in CHO cells.

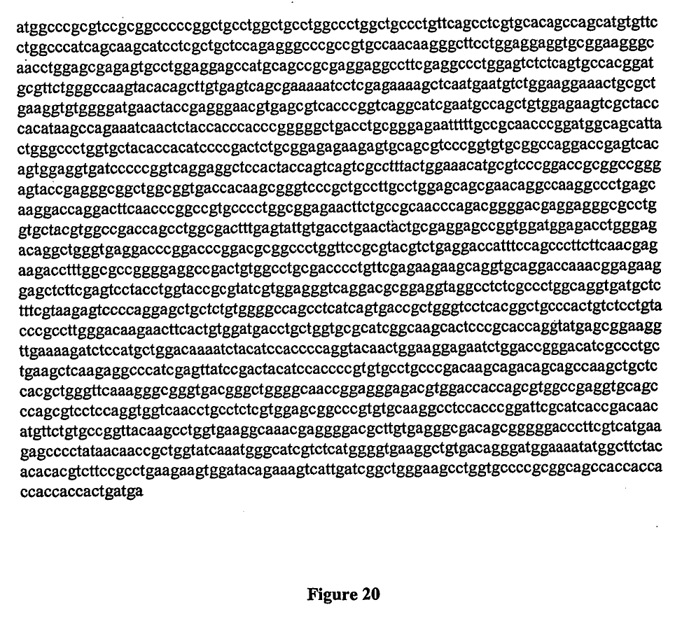

FIG. 21. Oligonucleotides/PCR primers used in mammalian cell expression cassettes.

For purposes of the present invention, as disclosed and claimed herein, the following terms are as defined below.

Signal Sequence: A DNA segment encoding a secretory peptide. Signal sequences may also be called leader sequences, and/or pre sequences. A secretory peptide is an amino acid sequence that acts to direct the secretion of a mature polypeptide or protein from a cell. Secretory peptides are typically characterized by a core of hydrophobic amino acids and are typically (but not exclusively) found at the amino termini of newly-synthesized proteins. The secretory peptide is cleaved from the mature protein during secretion. Such secretory peptides contain processing sites that allow cleavage of the secretory peptide from the mature protein as it passes through the secretory pathway. Processing sites may be encoded within the secretory peptide or may be added to the peptide by, for example, in vitro mutagenesis.

Pro sequence: A DNA segment which encodes a propeptide and functions to direct or signal the processing of a protein or peptide. Pro sequences may be preceded by a pre sequence and can be removed from the protein during processing.

An “isolated nucleic acid” is a nucleic acid molecule that is identified and separated from at least one contaminant nucleic acid molecule with which it is ordinarily associated in the natural source of the nucleic acid. Such an isolated nucleic acid molecule is other than in the form or setting in which it is found in nature. Isolated nucleic acid molecules therefore are distinguished from the nucleic acid molecule as it exists in natural cells.

Thrombin: A two chain, disulfide-bonded, glycosylated polypeptide that cleaves specific bonds in fibrinogen to produce fibrin monomers that self-assemble to form a fibrin clot.

Expression Vector: A DNA molecule which contains, a DNA sequence encoding a protein of interest together with a promoter and other sequences, such as a transcription terminator and polyadenylation signal, that facilitate expression of the protein. Expression vectors further contain genetic information that provides for their replication in a host cell, either by autonomous replication or by integration into the host genome. It will be evident to one skilled in the art that such information that provides for the autonomous replication of an expression vector in a host cell encompasses known mammalian and bacterial origins of replication. As is discussed in more detail herein, bacterial and mammalian expression vectors generally contain a bacterial origin of replication. Examples of expression vectors commonly used for recombinant DNA are plasmids and certain viruses, although they may contain elements of both. They also may include one or more selectable markers.

Transfection or transformation: The process of stably and hereditably altering the genotype of a recipient cell or microorganism by the introduction of purified DNA. This is typically detected by a change in the phenotype of the recipient organism. The term “transformation” is generally applied to microorganisms, while “transfection” is used to describe this process in cells derived from multicellular organisms.

DNA Construct: A DNA molecule, or a clone of such a molecule, either single or double-stranded, which has been modified through human intervention to contain segments of DNA combined and juxtaposed in a manner that would not otherwise exist in nature. DNA constructs may contain operably linked elements which direct the transcription and translation of DNA sequence encoding polypeptides of interest. Such elements include promoters, enhancers and transcription terminators. By virtue of the elements contained within the DNA constructs, certain constructs are understood to be capable of directing the expression and secretion of the encoded polypeptides. If a DNA sequence encoding a polypeptide of interest contains a secretory signal sequence, the DNA construct containing appropriate elements will be considered to be capable of directing the secretion of the polypeptide.

Fusion polypeptides or fusion proteins as used herein comprise polypeptides, protein fragments, variants, or derivatives fused to a heterologous peptide or protein. The fusion site generally contains a cleavage site that is cleavable by a specific enzyme such as thrombin. Heterologous peptides and proteins include, but are not limited to: an epitope to allow for detection and/or isolation of the fusion peptide; a transmembrane receptor protein or a portion thereof, such as an extracellular domain; or a transmembrane and intracellular domain; a ligand or a portion thereof which binds to a transmembrane receptor protein; a ligand or a portion thereof which is catalytically active; a protein or peptide which promotes oligomerization, such as leucine zipper domain; and a protein or peptide which increases stability, such as an immunoglobulin constant region (Fc fusion protein).

Protein C derivative(s) refer(s) to the recombinantly produced derivatives that differ from wild-type human protein C but when activated retain the essential properties i.e., proteolytic, amidolytic, esterolytic, and biological (anti-coagulant, anti-inflammatory, pro-fibrinolytic activities). The definition of human protein C derivatives as used herein also includes the activated form.

An object of the present invention is to provide methods for producing thrombin using recombinant methods. A feature of the present invention is the use of an expression vector comprising a DNA sequence encoding prothrombin. An additional feature of the present invention is the use of an expression vector comprising a DNA sequence encoding prethrombin-2. Another feature of the present invention is the use of expression vectors within host cells to produce engineered thrombin precursors that are either auto-activated in vitro or secreted in an activated form, thus not requiring Factor Xa processing.

Suitable host cells for cloning or expressing the nucleic acid (e.g., DNA) in the vectors herein include prokaryote, yeast, or higher eukaryote cells. Suitable prokaryotes include but are not limited to eubacteria, such as Gram-negative or Gram-positive organisms, for example, Enterobacteriacea such as E. coli. Various E. coli strains are publicly available, such as E. coli K12 strain MM294 (ATCC 3 1.446); E. coli X1 776 (ATCC 3 1.537); E. coli strain W3 110 (ATCC 27.325) and K5 772 (ATCC 53.635). Other suitable prokaryotic host cells include Enterobacteriaceae such as Escherichia, e.g., E. coli, Enterobacter, Erwinia, Klebisella, Proteus, Salmonella, e.g., Salmonella typhimurium, Serratia, e.g., Serratia marcescans, and Shigelila, as well as Bacilli such as B. subtilis and B. licheniformis (e.g., B. licheniformis 4 1 P disclosed in DD266,7 10, published 12 Apr. 1989), Pseudomonas such as P. aeruginosa, and Streptomyces. These examples are illustrative rather than limiting.

An embodiment of the present invention is the cloning and expression of recombinant bovine thrombin precursors in E. coli for the evaluation of the recombinant enzyme as a potential replacement for the animal-derived enzyme currently used in the biotechnology industry. The enzymes are produced in this microbial system and subsequently activated in vitro using thrombin. The protein is produced as inclusion bodies that require refolding to obtain properly folded material capable of self-activation. The nucleotide sequence is as shown in SEQ ID. NO:1 and the amino acid sequence is as shown in SEQ ID NO:2.

The bovine thrombin cDNA codes for a 625 amino acid peptide that consists of a 43 residue leader sequence (for secretion) and a 582 residue prothrombin peptide. Cloning of the cDNA sequence coding for the bovine preprothrombin gene was accomplished by PCR amplification of the sequence using bovine liver cDNA. The potential for PCR related sequence changes was addressed by analyzing multiple independently amplified and cloned DNA sequences. The primers presented in FIG. 3 were designed to amplify the cDNA sequences coding for several precursors to thrombin including the preprothrombin, prothrombin and prethrombin-2 molecules. Since the full-length cDNA sequence (preprothrombin) is approximately 2.1 Kb in length, the sequences were also amplified in smaller “fragments” in order to facilitate the amplification and cloning reactions. These cDNA “fragments” also provided additional independently amplified sequences for consensus sequence derivation.

The E. coli expression systems are designed to accept NdeI (5′ restriction site) to BamHI (3′ restriction site) gene cassettes. As a result, PCR primers are designed to incorporate these sites into the amplified products. In some cases, these sites are found naturally within an amplified sequence and thus have to be removed. It is also beneficial to have unique internal restriction sites for potential future engineering purposes. Based on the published sequence (genbank accession #J00041), there were naturally occurring BamHI and BglII restriction sites in the cDNA, which were deleted during the cloning process. In addition, a unique XhoI site was introduced for engineering of the signal sequence, if needed, for secretion purposes. Because these sites were needed for subsequent engineering and cloning purposes, these sites are removed and/or introduced (without altering the amino acid sequence). Thus the primers synthesized to amplify the gene in smaller fragments, are designed to introduce these “silent” nucleotide changes. Specifically, these changes included the introduction of the XhoI site into the 5′ region and removal of a naturally occurring BamHI site and one of two BglII sites (3′ region), which facilitates the modification of the 3′ terminus of the molecule.

The independently amplified cDNA sequences and “fragments” are cloned directly into TA cloning vectors and the cloned sequences are analyzed. The cloned sequences are then aligned to derive a consensus sequence. Comparison of the consensus sequence derived from the independently amplified clones against the Genbank sequence shows that there were numerous nucleotide differences. Of these nucleotide sequence differences, only three amino acid changes resulted (positions shown as bold in FIG. 5) compared to the published sequence. All three of the residues that changed were present in the “pro” portion of the protein and one of the differences (R95K) was a conservative change. The other two amino acid changes represent fairly dramatic alterations in terms of amino acid chemistry. In both cases it is a basic amino acid (histidine) that is altered and in one case the alteration results in a neutral (polar) residue (H230S). In the third case, the basic residue is converted to an acidic residue (H248D). For each nucleotide position in the cDNA, at least three (and in the majority of the cases five or more) independently amplified fragments covering that position, are used to derive the consensus sequence. The resulting constructs are captured in FIG. 6.

Another embodiment of the present invention is the engineering of recombinant versions of the bovine thrombin precursor, prethrombin-2. The nucleic acid is as shown in SEQ ID NO:3 and the amino acid sequence is as shown in SEQ ID NO:4. Prethrombin-2 is the smallest single chain precursor to thrombin. It is a 314 amino acid protein that is naturally converted into the “A” and “B” chains of thrombin upon cleavage by Factor Xa. In order to efficiently build engineered versions of prethrombin-2 for expression studies, a “base” expression cassette is constructed using PCR methods, Example 2. The construct contained the natural cDNA sequence with the exceptions that the internal BamHI and BglII sites are removed, a naturally occurring 3′ region StuI restriction site is removed, and a 5′ region StuI site and SacI site are incorporated. Further expression changes, such as the addition of purification and detection “handles” (polyHis and IMAC sequences) are made by incorporation of these sequences into the flanking amplification primers.

The prothrombin coding cDNA sequence is amplified, using the primers described in FIG. 3, to generate the wild-type prethrombin-2 PCR expression cassette product (0.859 kb). The independently amplified, cloned (TA vector intermediates), sequence confirmed and subcloned prethrombin-2 expression cassette are placed in the appropriate expression host backgrounds and expressed. The prethrombin-2 protein expressed well above the levels of all native E. coli proteins. Subsequent quantitative analyses performed using an HPLC assay, show that these strains produced as much as 4 grams of prethrombin-2 per liter of bacterial culture.

Further embodiments of the present invention include modifications to the molecule that help primarily in the purification process. The addition of purification “handles” such as the IMAC and polyHis peptides are incorporated. PCR primers are used to introduce the specific purification “handles” by amplification utilizing the prethrombin-2 “base” expression cassette vector as the amplification template.

Additional embodiments of the present invention include the incorporation of an “activation site cassette” into the coding sequence such that alternative cleavage cassettes can easily and efficiently be exchanged. This allows the replacement and testing of alternatives to the natural Factor Xa activation sequence. It is significant to note that although there are two Factor Xa cleavage sequences in the prethrombin-2 expression cassette, FIG. 8, only the second or the “downstream” cleavage site is required to activate (cleave “A” chain from “B” chain) the molecule. The “activation site cassette” is constructed by using PCR overlap extension mutagenesis to introduce a unique SacI restriction site upstream (5′) of the second Factor Xa cleavage sequence (FIG. 4). A unique StuI restriction site is introduced downstream of the second Factor Xa cleavage site allowing oligonucleotide adaptors to be synthesized as SacI to StuI fragments containing the alternative cleavage sequences. This is accomplished by deletion, using PCR mutagenesis, a naturally occurring StuI site found in the 3′ region of the cassette. All changes described are introduced without altering the amino acid sequence. FIG. 4 shows the complete nucleotide sequence and its translated product of the modified prethrombin-2 expression cassette.

Furthermore, synthetic oligonucleotides containing the unique SacI and StuI restriction sites at the appropriate ends introduce the alternative cleavage sites. Five fragments representing four different protease cleavage sites are substituted into the sequence. These included two versions of the thrombin cleavage site and sites for the enterokinase, rhinoviral 2A and rhinoviral 3C proteases. (FIG. 9). The expression vectors containing the modified sequences that result from these substitution experiments are shown in FIG. 10. All of the alternatively activatable molecules expressed well in E. coli.

Another embodiment of the present invention is a thrombin precursor that could be activated by thrombin itself (in vitro auto-activation). Material expressed in this strain was isolated and taken through the refold process. The properly folded enzyme is then treated with a very low level thrombin “seed” and its ability to activate recombinant human Protein C is examined. Thus recombinant bovine thrombin precursor is produced in bacterial cells, isolated and activated using thrombin as a “seed” and activating recombinant protein C in a manner similar to the animal-derived enzyme.

A key observation that resulted from the bacterial expression work was that the post-translational modifications were not critical to the enzyme's activity since it is well established that E. coli is not capable of these complex post-translational modifications. Furthermore, bacterial expression of recombinant thrombin demonstrates the ability of thrombin-activatable thrombin precursors to be activated to enzymatic activity.

In addition to prokaryotes, eukaryotic microbes such as filamentous fungi or yeast are suitable cloning or expression hosts for thrombin precursor vectors. Saccharomyces cerevisiae is a commonly used lower eukaryotic host microorganism. Others include Schizosaccharomyces pombe [Beach and Nurse, Nature 290: 140-3 (1981); EP 139,383 published 2 May 1995]; Muyveromyces hosts [U.S. Pat. No. 4,943,529; Fleer et al., Bio/Technology 9(10): 968-75 (1991)] such as, e.g., K lactis (MW98-8C, CBS683, CBS4574) [de Louvencourt et al., J. Bacteriol. 154(2): 737-42 (1983)]; K. fiagilis (ATCC 12,424), K. bulgaricus (ATCC 16,045), K wickeramii (ATCC 24,178), K waltii (ATCC 56,500), K. drosophilarum (ATCC 36.906) [Van den Berg et al., Bio/Technology 8(2): 135-9 (1990)]; K. thermotolerans, and K. marxianus; yarrowia (EP 402,226); Pichia pastoris (EP 183,070) [Sreekrishna et al., J. Basic Microbiol. 28(4): 265-78 (1988)]; Candid; Trichoderma reesia (EP 244,234); Neurospora crassa [Case et al., Proc. Natl. Acad Sci. USA 76(10): 5259-63 (1979)]; Schwanniomyces such as Schwanniomyces occidentulis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans [Ballance et al., Biochem. Biophys. Res. Comm. 112(1): 284-9 (1983)]; Tilbum et al., Gene 26(2-3): 205-21 (1983); Yelton et al., Proc. Natl. Acad. Sci. USA 81(5): 1470-4 (1984)] and A. niger [Kelly and Hynes, EMBO J. 4(2): 475-9 (1985)]. Methylotropic yeasts are selected from the genera consisting of Hansenula, Candida, Kloeckera, Pichia, Saccharomyces, Torulopsis, and Rhodotoruia. A list of specific species that are exemplary of this class of yeast may be found in C. Antony, The Biochemistry of Methylotrophs 269 (1982).

Suitable host cells for the expression of glycosylated thrombin precursors are derived from multicellular organisms. Examples of invertebrate cells include insect cells such as Drosophila S2 and Spodoptera Sp, Spodoptera high5 as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line [293 or 293 cells subcloned for growth in suspension culture, Graham et al., J. Gen Virol., 36(1): 59-74 (1977)]; Chinese hamster ovary cells/±DHFR (DG44, K1, DuxB11) [CHO, Urlaub and Chasin, Proc. Natl. Acad. Sci. USA, 77(7): 4216-20 (1980)]; mouse sertoli cells [TM4, Mather, Biol. Reprod. 23(1): 243-52 (1980)]; human lung cells (W138. ATCC CCL 75); human liver cells (Hep G2, HB 8065); and mouse mammary tumor (MMT 060562, ATCC CCL51). The selection of the appropriate host cell is deemed to be within the skill in the art.

Thus, another embodiment of the present invention is mammalian cell culture expression of bovine thrombin precursor molecules. The primary benefit of expression in mammalian systems is the ability to obtain more complex post-translational modifications. Another such benefit attributed to the use of eucaryotic expression systems is the formation of soluble and properly folded products. Additionally, the protein expressed in mammalian cells is secreted into the culture medium thereby providing for efficient purification.

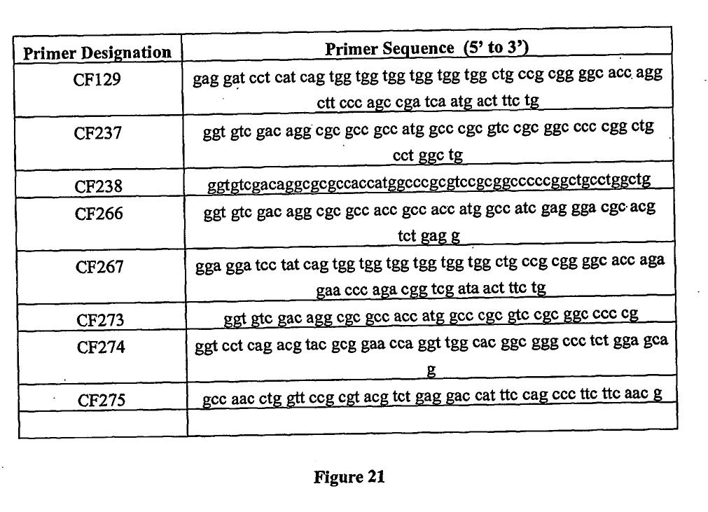

A preferred mammalian host cell line is the Chinese Hamster Ovary (CHO) cell line. More specifically, a DHFR mutant cell line designated DXB11 and an expression system (EASE) allowing for rapid generation of bulk expression cultures based on DHFR selection is utilized. The engineered versions of prothrombin (1.890 Kb) and prethrombin-2 (0.983 Kb) expression cassettes are generated by PCR amplification using the PCR primers shown in FIG. 22 and cloned directly into the EASE expression vector (pDC312). FIG. 13 describes the expression vectors constructed. Prothrombin and prethrombin-2 expression vectors are then transfected into the DXB11 parental cell line to generate CHO cell lines that secrete these thrombin precursors.

Prothrombin is purified using a two step process involving a Phenyl Sepharose column followed by an IMAC step. This purified protein is greater than 85% pure as determined by gel densitometry analyses. Prethrombin-2 is purified with a two step purification process involving the use of a SP Sepharose column followed by a Heparin affinity chromatography step. The resulting protein is greater than 85% pure.

The prothrombin precursor, is examined for its ability to be activated in vitro. Unexpectedly, the purified prothrombin precursor is activated in the absence of exogenous thrombin being added to the incubation reaction. Thus, the recombinant protein is auto-activated.

For prethrombin-2 expression cassette, the native preprothrombin leader sequence is fused to the prethrombin-2 sequence. Approximately 50% of the material expressed in the cells was secreted into the medium. In addition, the prethrombin-2 precursor molecule was activated in the culture medium, thus not requiring activation in vitro. The nucleic acid sequence is as shown in SEQ ID NO:7 and the amino acid sequence is as shown in SEQ ID NO:8.

Expression levels are monitored using the S2238 substrate assay, as well as, a thrombin ELISA. Surprisingly, the activation of prethrombin-2 precursor occurs at the time of secretion, since no fully intact precursor could be observed in the culture supernatant.

Standard kinetic analysis is performed to determine the catalytic characteristics of activated thrombin prepared from prothrombin and prethrombin-2 precursors. There is no significant difference between the Km values or specific activity on the S2238 substrate for thrombin prepared from either precursor molecule or commercially available native thrombin.

Additionally, individual thermal and pH stabilities are compared using a chromogenic substrate. The recombinant enzymes are equivalent to the animal derived enzyme across a wide range of temperature and pH.

Furthermore, experiments are carried out to evaluate the ability of the purified recombinant thrombin precursor molecules to activate protein C. The activated thrombin derived from recombinant thrombin precursor molecules activates recombinant protein C in a manner quite similar to the animal source enzyme.

Having generally described the invention, the same will be more readily understood by reference to the following examples, which are provided by way of illustration and are not intended as limiting.

### EXAMPLE 1

### Expression of Thrombin Precursor Molecules in Bacterial Cells

Bacterial Strains: The genotypes of the various bacterial strains that were used for this work are described in Table 1.

TABLE 1

| Strain Designation | Strain Genotype |
|---|---|
| DH5□ | endA1, hsdR17, supE44, thi1, recA1, gyrA, relA1, |
| | □(lacIZY A-argF, U169deoR(□80dlac□(lacZ)M15) |
| RV308 | RVlac□ₓ 74, gal ISII:OP308, strA |
| RQ228 | RV308 (Lac+)lacIq1, galE::P_{lacUV5} T7 RNAP (Km^{r}) |
| HMS174(DE3) | F⁻ recA1, hsdR17, Rif^{r} (DE3) |

DH5α served as the general “cloning” host since it was easily made competent and is amenable to passage of unmodified DNA because it is a restriction deficient yet modification positive (r−, m+) strain. Thus DNA synthesized in vitro (i.e. oligonucleotides used to create adaptors) could be passed through this host prior to being transformed into restriction positive expression hosts. RV308 served as the host of choice in these studies for use with the lambda expression system, which is temperature inducible. RQ228 represents an expression host specifically for use with the T7 and T7lac expression systems. HMS174(DE3) was a commercially available T7 expression host also used in these studies.

Media and growth conditions: Shake-flask bacterial cultures were routinely grown in LB medium (Miller 1972). L agar consisted of LB medium plus 15 g Bacto Agar (Difco) per liter. When appropriate, antibiotics (Sigina and BRL) were added at the following concentrations: chloramphenicol (25 ug/mL), tetracycline (12.5 ug/mL), ampicillin (100 ug/mL), kanamycin (50 ug/mL), nalidixic acid (20 ug/mL), neomycin (75 ug/mL) and streptomycin (50 ug/mL). For T7 expression system induction experiments, IPTG was made as a 0.5 M stock in water and added to the culture medium at a final concentration of 0.1 mM to 1 mM.

### EXAMPLE 2

### DNA Methods

Plasmid DNA was isolated using either the Wizard Purification kit (Promega Corp.) or the Plasmid DNA Spin Column kit (Qiagen). Agarose gel electrophoresis of DNA, “filling-in” of restriction fragments with protruding 5′ ends by Klenow, ligation of DNA fragments, and transformation of E. coli by calcium chloride method were carried out as described by Maniatis et. al. and/or according to Current Protocols Manual. Individual restriction fragments were isolated in low-melting-temperature agarose by the method of Struhl. (Struhl, K. 1985) or by use of a Qiagen DNA gel purification kit according to the manufacturer's recommendations.

Cloning and Expression Vectors: The various expression vector backbones utilized in this work are presented in FIG. 11. The key characteristics of each of these constructs are also presented in the table, including the specific promoter system they possess, as well as the method used to induce expression when these backbones were utilized.

PCR and adaptor oligonucleotides: The oligonucleotides that were designed and synthesized for use are listed in FIG. 3. The table also distinguishes between the specific use(s) for the oligonucleotides. One set of the primers were designed based on the known sequence for bovine thrombin as derived from the original Genbank submission #J00041. These particular oligonucleotides were designed to allow for the PCR amplification of multiple versions and fragments of the bovine thrombin cDNA. Versions included the full-length preprothrombin, prothrombin and prethrombin-2 precursor molecules. These primers were also used to amplify internal fragments for each of these versions. This was done in order to provide additional independently amplified sequences for comparison to the expected sequence, as well as for the purpose of making certain modifications to the sequences such as insertion and/or removal of specific restriction sites. PCR primers were used to generate the consensus sequence for the cloned cDNA and build the initial framework for subsequent cloning and expression work. Subsequently, PCR primers were used to make additional engineering modifications to the sequences (i.e. restriction site changes, amino acid alterations, purification handles etc). FIG. 12 shows the specific nucleotide sequences that were generated to incorporate the handle on the 5′ and 3′ termini of the expression cassette, respectively. All oligonucleotides (for both linker addition cloning and for use as PCR primers) were synthesized and purified at 0.2 umole scale by Genosys, Inc.

PCR amplification and cDNA cloning: PCR primers were diluted to make stock concentrations of 50 uM. PCR reactions were run in Perkin Elmer Geneamp PCR system 9600 thermocycler. PCR Amplification was performed as follows: templates consisted of either freshly isolated cells taken from an isolated colony on agar plate or diluted purified plasmid DNA (1 ng/uL final concentration diluted in water). In the case of the initial cDNA (Clontech) cloning, the commercially purchased cDNA (1 ug) was used directly as the amplification template. In the case where cells were directly used, the cells were lysed by heating them to 99.9° C. for 10 minutes prior to 32 cycles of the following cycle: 94° C. for 30 seconds, 58° C. for 30 seconds, 72° C. for 75 seconds. The regular cycling program was followed by a 10 minute 72° C. extension step and the sample was then held at 4° C. until subsequent use. Direct cloning of the PCR products was accomplished utilizing the TA cloning kit (pCRII or pCR2.1Topo vectors) according to the manufacturer's directions. The sequence integrity of all constructs generated by either PCR or direct ligation of synthetic DNA oligonucleotides (i.e. adaptor cloning) was confirmed by sequence analyses. In the case of the initial cloning of the cDNA for bovine thrombin, at least three independently amplified clones were obtained to represent each base position. This provided a means of identifying a consensus sequence for the cloned coding DNA sequence.

### EXAMPLE 3

### Activation of Recombinant Thrombin using Snake Venom

This procedure was carried out essentially as described by DiBella et aL, (J. Biol. Chem., 270(1): 163-169,1995). Snake venom (E. carinatus snake venom, Sigma #V-8250) was pretreated with p-APMSF to inactivate trypsin-like proteases (Laura et al., 1980). The protein concentration of this treated snake venom preparation was determined with a BCA assay. Activations were then set up using a prethrombin-2 to snake venom weight ratio of 5:1 in a reaction mixture containing 25 mM sodium phosphate (monobasic), pH 7.4 and 0.4 M NaCl. Reactions were incubated at 37 degrees C. Aliquots were removed at 30 minute intervals and assayed using the S2238 spectroscopic assay according to the manufacturer's recommendations (Chromogenix Inc.). Typical activations reached their maximum activity in 2-3 hrs under these conditions.

### EXAMPLE 4

### Thrombin S2238 Substrate Activity Assay

The S2238 chromogenic substrate was purchased from Chromogenix, Inc. and was used according to the manufacturer's protocol. Briefly, control samples were made by dissolving bovine thrombin in Tris buffered saline (20 mM Tris/150 mM NaCl)/10 mM EDTA pH 8.3 containing 0.1 mg/ml BSA to a final TBS concentration of approximately 1 mg/ml. The protein concentration was estimated using E^{1%}=19.5 at 280 nm (Winzor and Scheraga, 1964). Recombinant thrombin or control samples were diluted with the TBS/EDTA/BSA buffer to a final protein concentration of 1 μg/ml. Approximately 100 μl of each sample was added to plastic cuvettes containing 100 μM S2238 (a synthetic peptide substrate with a p-nitroaniline leaving group) in TBS/EDTA/BSA buffer pre-equilibrated of 37° C. for 10 minutes in a spectrophotometer set at 405 nm. The increase in the absorbance at 405 nm was measured over 5 minutes with data acquired every 15 seconds. The change in absorbance per minute was divided by 1.67 to calculate NIH units of activity. This number was divided by the protein concentration used in the assay to calculate the specific activity (in NIH units/mg protein) of the enzyme.

### EXAMPLE 5

### Autoactivation using Thrombin

Activation using thrombin is similar to the reactions described above using snake venom. Initial activations were set up using a prethrombin-2 to bovine thrombin weight ratio of 100:1 in a reaction mixture containing 20 mM Tris pH 8.3, 0.15 M NaCl, 10 mM EDTA. Reactions were incubated at 37° C. Aliquots were removed at 30 minute intervals and assayed using the S2238 spectroscopic assay. Typical activations reached their maximum activity in 1-1.5 hrs under these conditions.

### EXAMPLE 6

### Purification of Recombinant of E. Coli Prethrombin-2

The desired expression plasmid/host was grown in shake-flask volumes up to several liters (250 mL/1 liter flasks) shaking (290 rpm) at 37° C. The growth was monitored by following OD600 and samples were taken prior to addition of IPTG (100 uM) and following continued growth (4-6 hours) and eventual harvest. The cell pellet was obtained by centrifugation (GSA rotor in Sorval centrifuge) at 6500 rpm for 10 minutes. The spent medium was decanted and the pellet was resuspended in 7 M gaunidine-HCl/10 mM Tris pH 8.0 in order to lyse the cells. The insoluble fraction containing the prethrombin-2 inclusion bodies were isolated by high-speed centrifugation (20 k rpm) and the soluble fraction was decanted. The inclusion bodies were then sulfonated as described by Dibella et al.

Purification of Recombinant Sulfonated Prethrombin-2:

The sulfonated prethrombin-2 was dissolved in 7 M guanidine-HCl/10 mM Tris pH 8.0 and loaded onto a semipreparitive Vydac C-18 column (1.0×25 cm) with a linear flow rate of 75 cm/hr. The column was then eluted with a 4 column volume (CV) gradient from 0-70% buffer B (buffer A=23% ACN/0.1% TFA, buffer B=90% ACN/0.1% TFA). Fractions containing the prethrombin-2 (as determined by analytical HPLC) were pooled and lyophilized.

### EXAMPLE 7

### Dodecapeptide (DDP) Release Assay

Briefly, samples containing thrombin-like activity were incubated with human Protein C at a final protein concentration of 2 mg/ml in 20 mM Tris/150 mM NaCl pH 8.0. The final reaction volume was 2 ml. The reaction was then adjusted to pH 6.0 with 1 N citric acid and incubated at 40° C. for the indicated times. A bovine thrombin control sample was set up in the same way using approximately 180 ug of enzyme activity previously dissolved in 20 mM Tris/150 mM NaCl pH 8.0 at a final concentration of 2 mg/ml. Reaction aliquots (25 μl) were removed and quenched with 25 μl of 0.1% TFA. Samples were analyzed for the release of dodecapeptide using a Shandon Hypercarb column (3.2×100 mm) attached to a HP1090 HPLC equipped with an external Applied Biosystems detector set at 216 nm. Released dodecapeptide was quantitated against a standard curve using chemically synthesized authentic dodecapeptide.

### EXAMPLE 8

### Mammalian Cell Culture

The mammalian cells used in these experiments are Chinese Hamster Ovary. (CHO) cells and specifically the DXBll line. This particular cell line is a DihydroFolate Reductase (DHRF) mutant. This mutation allowed for the selection of DHFR-containing cells using hypoxathine/thymidine minus (−HT) media, with methotrexate addition increasing amplified titers.

The cells were grown in suspension in serum-free maintenance media, which consisted of Excell302 (JRH) containing 4 mM L-Glutamine (BRL) and 1× HT supplement (BRL). Following transfection, cells were placed in selective Excell302 medium containing 4-8 mM L-Glutamine and 1× dextran sulfate (Sigma). In some cases, MTX (Sigma) was added to either 50, 150 or 300 nM from an appropriate stock solution made up in DMSO (Sigma).

### EXAMPLE 9

### Design of the Mammalian Expression Cassette

The expression system was obtained from Immunex and is called the Expression Augmenting Sequence Element or EASE system. This system is reported to allow the development of a stable, high-expressing CHO cell “pool” in a rapid time frame (Aldrich et al., Cytotechnology, 28:1-9, 1999). The EASE vector is proprietary and the complete sequence is thus not available. The vector did come with a multi-cloning site region, which contained restriction sites for the subcloning of the gene of interest. Since the bacterial expression cassettes are on the NdeI to BamHI restriction fragment, the BamHI site at the 3′ end of the gene is utilized. A SalI site at the 5′ end of the gene is introduced by PCR and the expression cassette is then subcloned into the EASE vector as a SalI to BamHI fragment. The oligonucleotides used as PCR primers in the construction of the prothrombin and prethrombin-2 expression constructs are listed in (FIG. 22). The PCR reactions were carried out in the Perkin Elmer Cetus GeneAmp 9600 thermocycler using the following parameters: initial template denaturation at 98° C. for 10 minutes followed by 30 cycles at 94° C. for 30 seconds, 56° C. for 30 seconds and 72° C. for 3 minutes. The final cycle was followed by a 72° C. 10 minute extension and then the reactions were held at 4° C. until they could be further processed.

Mammalian expression cassette are constructed utilizing specific cloning sites described above (i.e. SalI to BamHI cassettes) and the kozak sequence. The kozak sequence is a short region that is reported to be important in translational intitation in eucaryotes. The consensus sequence for the Kozak region is as follows: GCCG/ACC. Both regions of the consensus Kozak sequence (Kozak, 1981) are included to determine if there was any significant differences in expression between them. A COOH-terminal sequence is incorporated as a “tag” for diagnostic purposes (expression analyses) and to provide a potential “handle” for purification purposes.

### EXAMPLE 10

### Transfection (Electroporation)/Selection/Amplification

Transfection is accomplished using GenePulsar II (Biorad) equipment according to the following protocol:

For each transfection sample 10 ug DNA is linearized using a restriction enzyme (PvuI) that cuts within the ampicillin resistance gene, but not in portions of the vector that would be essential to mammalian cell selection or expression. The DNA is digested overnight at 37° C. and is subsequently ethanol precipitated and washed prior to allowing the DNA pellet to dry in the hood for 20 minutes.

Cells were prepared from a parental cell culture that has been passaged for a minimum of three times, but no more than 30 generations from a frozen vial. Cells are grown at 37° C. in 5% CO2 in Excell 302 medium (JRH) supplemented with L-glutamine to a final concentration of 4 mM and containing 1× HT supplement. 2-3 days prior to transfection the parental cell line is subcultured and seeded to a final density of 0.25×10e6/mL. On the day of the transfection the cells are collected at a density of between 0.7-1.0×10e6/mL. Cells are harvested by centrifugation (1500 rpm) and resuspended in an amount of medium to bring the final cell density to 1×10e7 per 800 uL. 800 ul of cells are placed in a 0.4 cm gap electroporation cuvette. The cuvette is placed on ice for 10 minutes.

The Gene Pulsar II unit is set at 350 volts and 925 uF. The sample is inserted and the electrical pulse is delivered according to the manufacturer's recommendations. The sample is removed and placed on ice for 10 minutes. Both the actual voltage delivered and time constant are recorded to provide an indication of the effectiveness of the pulse delivered. Following the post-electroporation incubation on ice, the samples are removed from the cuvettes and placed into T75 flask and incubated for 48 hours undisturbed at 37° C. and 5% CO2. After recovery, the samples are counted and the viability examined prior to placing the samples under selection.

Initial Selection: The EASE vector contains the DHFR gene and thus selection is based on this marker. Two selection/amplification schemes were used in the generation of cell lines. In both schemes, the cells were cultured in “bulk suspension” (starting from 20 mL cultures in 250 mL shake flasks) and there was no attempt to obtain single cell clones or otherwise homogeneous populations. One scheme involved initial selection by growing the recovered cells in Excell 302+ L-Glutamine in the absence of the HT supplement. This selection is referred to as “−HT” or “basal” selection. In this case the cells are cultured in bulk (maintaining a viable cell density of 0.2×10e6/mL) in the appropriate volumes and vessels, until the population reaches a viability of >90%. At which time the lines are cryopreserved, analyzed for expression and taken into the amplification process.

The second selection scheme involves placing the post-transfected recovered population directly into several levels of MIX selection. These levels are 50, 150 and 300 nM. Cells were again cultured as described above until they reached a stable cell viability, preferably >90%, but in some cases they stabilized below this level.

Amplification Protocol: Amplification is based on the presumption that any given population of cells contains heterogeneity. This heterogeneity exists at the chromosomal level. Thus by placing a population under increasing levels of a selective agent, the cells within the population that possess more copies of the resistance marker are those cells that are selected for and eventually will grow out from the selection conditions. The resistance marker should be linked genetically to the gene of interest (in this case thrombin), and thus amplification results in higher copies of the expression cassette, which in turn results in higher expression levels. Amplification is not a general process and thus is not possible for any given resistance marker. Amplification based on the MTX marker is well established, however neomycin resistance is not amenable to amplification.

The protocol for amplification is performed in bulk culture and is carried out by seeding a stabilized “parental” cell line at 5×10e5 cells/mL in a 20 mL shake flask volume using the appropriate selective medium. The culture is subcultured every 3-4 days, measuring cell viability and viable cell densities at each pass. The culture is maintained at the original seeding density and thus the overall culture volume may be decreased by the harvest and resuspension of the cells. Eventually, the resistant cell population will begin to grow out and the volume will be increased as the cells stabilize under the new selective conditions. Once the culture has stabilized, expression analyses can take place and samples can be preserved for long-term storage.

### EXAMPLE 11

### Shake-Flask Exression Analyses

All expression analyses was performed on bulk cultures grown in shake-flask scale. Expression was followed primarily by 4-12% Bis-Tris SDS-PAGE (Invitrogen) and subsequent Western blot analyses. Both thrombin and anti-His antibodies were utilized according to established. In some cases where active thrombin was present, an ELISA is utilized to determine titers.

Conditioned medium was used either directly or concentrated using Microcon-30 microconcentrators (Amicon). For intracellular analyses, cells were collected by centrifugation (1500 rpm), washed using 1× PBS and resuspended in TPER lysis buffer (Pierce). Cell lysates were then separated from the cell debris by centrifugation again and the lysates analyzed by SDS-PAGE and Western blot as described below.

SDS polyacrylamide gel electrophoresis: Samples were analyzed by 4-12% Bis-Tris NuPAGE gels using MOPS running buffer. Samples were diluted into 4× NUPAGE LDS (lithium dodecyl sulfate) reducing sample buffer. The samples were then heated using a heat block at 85° C. for 10 minutes. A measured aliquot of each heated sample was loaded onto the gel(s) and electrophoresis was carried out at a constant voltage of 200 volts. Gels were stained with colloidal blue or silver stain. Low molecular weight standards (BioRad) were used: phosphorylase b (97 kDa), BSA (66 kDa), ovalbumin (45 kDa), carbonic anhydrase (31 kDa), trypsin inhibitor (22 kDa) and lysozyme (14 kDa).

### EXAMPLE 12

### Prothrombin Purification

Purified recombinant prothrombin was obtained by a two step process involving an initial Phenyl-Sepharose capture followed by Ni—NTA Immobilized Metal Affinity Chromatography (IMAC). This was accomplished by collecting conditioned media containing recombinant bovine prothrombin from the appropriate CHO cell line. Centrifugation (Sorvall/GSA rotor/8000 rpm/10 minutes) was used to separate cells from the spent medium.

The Phenyl Sepharose (Hi sub) purification step was carried out as follows: solid NaCl was added (to a final concentration of 2 M) to 3 L of conditioned media, pH 6.4, and charged directly onto a 1.6×16.5 cm (33 ml) Phenyl Sepharose (high sub). After charging, the column was washed for 3.5 column volumes with 20 mM phosphate, pH 7, 2 M NaCl followed by a 2 column volume gradient from 2-0.5 M NaCl followed by a 5 column volumes 0.5-0 M NaCl gradient. A final 2 column volume 0.5 M NaCl wash was also performed. All elution buffers contained 20 mM phosphate buffer at pH 7.0. Fractions from both gradient steps were analyzed by Western blotting using anti-thrombin and anti-His antibodies. Fractions containing recombinant prothrombin were then pooled for use in the next purification step.

The IMAC chromatography step was then performed as follows: imidazole was added to the Phenyl mainstream at a final concentration of 17 mM before charging onto a 4 ml (1.1×4 cm) Ni—NTA superflow (Qiagen) column. The flow rate throughout was 0.8 ml/min. After loading was complete, the column was washed with 3.5 column volumes (cv) of 20 mM phosphate, pH 7, 0.5 M NaCl, 15 mM imidazole, and 0.1% polyethylene glycol (PEG) 8000. The target protein was eluted by a 8 cv 15-300 mM imidazole gradient in the presence of 20 mM phosphate, pH 7, 0.05 M NaCl, and 0.1% PEG 8000. Fractions (2 ml) were collected during the gradient and analyzed by reducing SDS-PAGE and western blot. Fractions were pooled based on quantity and purity of IMAC tagged recombinant prothrombin by SDS-PAGE. All chromatography steps were performed in a chill room (4-8 C) on a Pharmacia FPLC.

### EXAMPLE 13

### Prethrombin-2 Purification

Cell lines expressing prethrombin-2 did not secrete the intact precursor molecule. In this case, a two step process is used that allows the isolation of the active form of bovine thrombin. This process consists of an ion exchange capture (e.g. SP Sepharose Fast Flow), followed by a Heparin affinity step. As is the case with prothrombin purification, material is isolated from conditioned medium in which the cells are separated from the medium by centrifugation (Sorval/GSA rotor/8000 rpm/10 minutes).

The SP Sepharose Fast Flow Chromatography step is carried out as follows: clarified conditioned media (900 mL) containing active thrombin was diluted with 50 mLs of 20 mM phosphate, pH 6.5. The sample is then diluted to a final volume of 2000 mL using distilled water to reduce conductivity (at 20 C) from 13.9 mmhos to 6.6 mmhos and a final pH 6.5. This material is then charged directly onto a 1.6×13 cm (26 mL) SP Sepharose Fast Flow column. The flow rate is 3 mL/min throughout the run. After loading is complete, the column is washed with 5 column volumes of 20 mM Phosphate pH 6.5. The target protein is eluted by a 10 column volume 0-0.6M NaCl gradient in the presence of 20 mM phosphate pH 6.5. Fractions (10 mL) are collected during the gradient and analyzed by S2238 enzymatic activity and reducing SDS-PAGE. Fractions are pooled based on activity and purity.

The Heparin affinity step is carried out as follows: The pooled fractions (70 mL) taken from the SP Sepharose step are diluted with 86 mL of 20 mM phosphate buffer pH 7.4 and 335 mL of distilled water resulting in a final conductivity of 6.9 mmhos and pH 6.9. The diluted material (470 mL) is charged onto a 1 mL pre-packed Heparin Hi-Trap (Pharmacia) at a flow rate of 3 mL/min. The column is washed with 20 column volumes of 20 mM phosphate pH 7.4 followed by a 50-column volume elution gradient 0-0.7M NaCl. Fractions (4 mL) are collected during the gradient and analyzed by S2238 enzymatic activity as well as reducing SDS-PAGE with silver staining to visualize the gel. The fraction containing the highest specific activity is used for the kinetic studies.

### EXAMPLE 14

### S2238 Activity Assay

The S2238 activity assay is used to determine the activity of bovine thrombin. The principle of this method is to measure the ability of thrombin to hydrolyze the substrate S-2238, H-D-Phenylalanyl-L-pipecolyl-L-arginine-p-nitrolanaline dihydrochloride. More specifically, thrombin catalyzes the hydrolysis of p-nitroanalide (pNA) from the peptide substrate S-2238. The rate at which the pNA is released is measured in a Beckmann DU620 spectrophotometer at 405 nm.

### EXAMPLE 15

### Protein C Activation Assay

This method is used to determine the relative specificity of bovine thrombin used in protein C activation. A reverse phase HPLC method is used to measure the formation of two activation peptides, dodecapeptide (residues #158-169), octadecapeptide (residues #152-169), and two additional peptides formed during the activation. Thrombin specificity is defined as the ratio between the area of the activation peptides and the total area of the peptides generated during activation. The relative specificity is defined as the ratio between the specificity of the tested thrombin and the specificity of the bovine thrombin reference standard.

Recombinant Protein C Activation: Stock solutions of dodecapeptide (DDP) and octadecapeptide (ODP) are prepared from lyophilized powders to 200 ug/mL using activation buffer as the diluent. These stocks are combined in equal volume to create a 100 ug/mL ODP and 100 ug/mL DDP standard that is injected onto the machine. The hPC reference standard is prepared by reconstituting one vial to 1 mg/mL using activation buffer. Bovine thrombin samples are prepared using reconstitution buffer to resuspend the powder enzyme to 1-2 mg/mL. The recombinant thrombin did not need to be resconstituted. The absorbance of the thrombin samples was read at OD260 and OD280. The absorbance data and chromogenic substrate assay data were used to confirm the actual concentration of the enzyme in the reaction. 5 Units of bovine thrombin sample was added to the reconstituted hPC. The samples were incubated at 37° C. for 6 hours with stirring. Following the incubation the samples were immediately stored at −70° C. until they could be injected onto the HPLC equipment. HPLC analyses was accomplished using a Zorbex 300SB-C18 reversed phase column run at 60° C. and a flow rate of 1.5 mL/min (4.6 mm×15 cm, 3.5 uM particle size). Detection was measured by absorbance at 216 nm to detect amide bonds. A TFA/ACN binary gradient was used for the elution column.

Activation of recombinant protein C derivative molecules: One of skill in the art realizes that the bovine thrombin precursor molecules of the present invention can also be used to activate derivatives or analogs of protein C. For example, human protein C derivatives S11G:Q32E:N33D and H10Q:S11G:Q32E:N33D.

Human protein C derivative S11G:Q32E:N33D contains a glycine residue at position 11 instead of a serine residue normally found at this position, a glutamic acid residue at position 32 instead of the glutamine residue normally found at this position an aspartic acid residue at position 33 instead of the asparagine residue normally found at this position.

Human protein C derivative H10Q:S11G:Q32E:N33D contains a glutamine residue at position 10 instead of the histidine residue normally found at that position, a glycine residue at position 11 instead of the serine residue normally found at this position, a glutamic acid residue at position 32 instead of the glutamine residue normally found at this position, and an aspartic acid residue at position 33 instead of the asparagine residue normally found at this position.

1. An isolated nucleic acid encoding a recombinant bovine thrombin precursor molecule wherein said precursor molecule is prothrombin and said isolated nucleic acid is as shown in SEQ ID NO:5.

2. A polynucleotide encoding a polypeptide having the amino acid sequence as shown in SEQ ID NO:6, wherein said polypeptide is the recombinant bovine thrombin precursor molecule prothrombin.

3. An isolated nucleic acid encoding a recombinant bovine thrombin precursor molecule wherein said precursor molecule is prethrombin-2 and said isolated nucleic acid is as shown in SEQ ID NO:7.

4. A polynucleotide encoding a polypeptide having the amino acid sequence as shown in SEQ ID NO:8, wherein said polypeptide is the recombinant bovine thrombin precursor molecule prethrombin-2.

5. An isolated nucleic acid encoding a recombinant bovine thrombin precursor molecule wherein said precursor molecule is prothrombin and said isolated nucleic acid is as shown in SEQ ID NO:1.

6. A polynucleotide encoding a polypeptide having the amino acid sequence as shown in SEQ ID NO:2, wherein said polypeptide is the recombinant bovine thrombin precursor molecule prothrombin.

7. An isolated nucleic acid encoding a recombinant bovine thrombin precursor molecule wherein said precursor molecule is prethrombin-2 and said isolated nucleic acid is as shown in SEQ ID NO:3.

8. A polynucleotide encoding a polypeptide having the amino acid sequence as shown in SEQ ID NO:4, wherein said polypeptide is the recombinant bovine thrombin precursor molecule prethrombin-2.

9. The precursor molecule of claims 1 to 8, wherein said precursor molecule is auto-activated.

10. A vector comprising the nucleic acid molecule of any of claims 1-4.

11. A vector comprising the nucleic acid molecule of any of claims 1-4, wherein said nucleic acid molecule is operably linked to control sequences recognized by a host cell transformed with said vector.

12. A host cell comprising a vector comprising the nucleic acid molecule of any of claims 1-4, wherein said nucleic acid molecule is operably linked to control sequences recognized by a host cell transformed with said vector and, wherein said host cell is a CHO cell.

13. A vector comprising the nucleic acid molecule of any of claims 5-8.

14. A vector comprising the nucleic acid molecule of any of claims 5-8, wherein said nucleic acid molecule is operably linked to control sequences recognized by a host cell transformed with said vector.

15. A host cell comprising a vector comprising the nucleic acid molecule of any of claims 5-8, wherein said nucleic acid molecule is operably linked to control sequences recognized by a host cell transformed with said vector and wherein said host cell is E. coli.

16. A process for purifying activated thrombin wherein said process consists of the steps:
(a) expressing recombinant bovine thrombin precursor molecule prethrombin-2 as shown in SEQ ID NO:8, in a host cell;
(b) purifying said recombinant bovine thrombin precursor molecule by ion exchange chromatography;
(c) further purifying said recombinant bovine thrombin precursor molecule by heparin affinity chromatography; and
(d) assaying said purified recombinant bovine thrombin precursor molecule for purity and thrombin activity.

17. The process of claim 16 wherein said host cell is CHO cells.

18. The process of claim 17 wherein said recombinant bovine thrombin precursor molecule is activated when expressed from said CHO cells as prethrombin-2.

19. A method of producing an activated protein wherein said method uses any of the recombinant thrombin precursor molecules of claims 1 to 8.

20. A method of producing an activated protein wherein said method uses any of the recombinant thrombin precursor molecules of claims 1 to 8, wherein said activated protein is protein C or a derivative thereof.

21. A method of producing an activated protein wherein said method uses any of the recombinant thrombin precursor molecules of claims 1 to 8, wherein said activated protein contains an activation sequence other than the native factor Xa sequence.


SEQUENCE LISTING (8 sequences)

SEQ ID NO: 1 - DNA, 1755 bp, Bovine engineered prothrombin
atggccaacaagggcttcctggaggaggtgcggaagggcaacctggagcgagagtgcctggaggagccatgcagccgcgaggaggccttcgaggccctggagtctctcagtgccacggatgcgttctgggccaagtacacagcttgtgagtcagcgaaaaatcctcgagaaaagctcaatgaatgtctggaaggaaactgcgctgaaggtgtggggatgaactaccgagggaacgtgagcgtcacccggtcaggcatcgaatgccagctgtggagaagtcgctacccacataagccagaaatcaactctaccacccacccgggggctgacctgcgggagaatttttgccgcaacccggatggcagcattactgggccctggtgctacaccacatccccgactctgcggagagaagagtgcagcgtcccggtgtgcggccaggaccgagtcacagtggaggtgatcccccggtcaggaggctccactaccagtcagtcgcctttactggaaacatgcgtcccggaccgcggccgggagtaccgagggcggctggcggtgaccacaagcgggtcccgctgccttgcctggagcagcgaacaggccaaggccctgagcaaggaccaggacttcaacccggccgtgcccctggcggagaacttctgccgcaacccagacggggacgaggagggcgcctggtgctacgtggccgaccagcctggcgactttgagtattgtgacctgaactactgcgaggagccggtggatggagacctgggagacaggctgggtgaggacccggacccggacgcggccctggttccgcgtacgtctgaggaccatttccagcccttcttcaacgagaagacctttggcgccggggaggccgactgtggcctgcgacccctgttcgagaagaagcaggtgcaggaccaaacggagaaggagctcttcgagtcctacctggtaccgcgtatcgtggagggtcaggacgcggaggtaggcctctcgccctggcaggtgatgctctttcgtaagagtccccaggagctgctctgtggggccagcctcatcagtgaccgctgggtcctcacggctgcccactgtctcctgtacccgccttgggacaagaacttcactgtggatgacctgctggtgcgcatcggcaagcactcccgcaccaggtatgagcggaaggttgaaaagatctccatgctggacaaaatctacatccaccccaggtacaactggaaggagaatctggaccgggacatcgccctgctgaagctcaagaggcccatcgagttatccgactacatccaccccgtgtgcctgcccgacaagcagacagcagccaagctgctccacgctgggttcaaagggcgggtgacgggctggggcaaccggagggagacgtggaccaccagcgtggccgaggtgcagcccagcgtcctccaggtggtcaacctgcctctcgtggagcggcccgtgtgcaaggcctccacccggattcgcatcaccgacaacatgttctgtgccggttacaagcctggtgaaggcaaacgaggggacgcttgtgagggcgacagcgggggacccttcgtcatgaagagcccctataacaaccgctggtatcaaatgggcatcgtctcatggggtgaaggctgtgacagggatggaaaatatggcttctacacacacgtcttccgcctgaagaagtggattcagaaagttatcgaccgtctgggttcttaatag

SEQ ID NO: 2 - protein, 583 aa, Bovine engineered prothrombin
MANKGFLEEVRKGNLERECLEEPCSREEAFEALESLSATDAFWAKYTACESAKNPREKLNECLEGNCAEGVGMNYRGNVSVTRSGIECQLWRSRYPHKPEINSTTHPGADLRENFCRNPDGSITGPWCYTTSPTLRREECSVPVCGQDRVTVEVIPRSGGSTTSQSPLLETCVPDRGREYRGRLAVTTSGSRCLAWSSEQAKALSKDQDFNPAVPLAENFCRNPDGDEEGAWCYVADQPGDFEYCDLNYCEEPVDGDLGDRLGEDPDPDAALVPRTSEDHFQPFFNEKTFGAGEADCGLRPLFEKKQVQDQTEKELFESYLVPRIVEGQDAEVGLSPWQVMLFRKSPQELLCGASLISDRWVLTAAHCLLYPPWDKNFTVDDLLVRIGKHSRTRYERKVEKISMLDKIYIHPRYNWKENLDRDIALLKLKRPIELSDYIHPVCLPDKQTAAKLLHAGFKGRVTGWGNRRETWTTSVAEVQPSVLQVVNLPLVERPVCKASTRIRITDNMFCAGYKPGEGKRGDACEGDSGGPFVMKSPYNNRWYQMGIVSWGEGCDRDGKYGFYTHVFRLKKWIQKVIDRLGS

SEQ ID NO: 3 - DNA, 948 bp, Bovine engineered prethrombin-2
atggccatcgagggacgcacgtctgaggaccatttccagcccttcttcaacgagaagacctttggcgccggggaggccgactgtggcctgcgacccctgttcgagaagaagcaggtgcaggaccaaacggagaaggagctcttcgagtcctacctggtaccgcgtatcgtggagggtcaggacgcggaggtaggcctctcgccctggcaggtgatgctctttcgtaagagtccccaggagctgctctgtggggccagcctcatcagtgaccgctgggtcctcacggctgcccactgtctcctgtacccgccttgggacaagaacttcactgtggatgacctgctggtgcgcatcggcaagcactcccgcaccaggtatgagcggaaggttgaaaagatctccatgctggacaaaatctacatccaccccaggtacaactggaaggagaatctggaccgggacatcgccctgttgaagctcaagaggcccatcgagttatccgactacatccaccccgtgtgcctgcccgacaagcagacagcagccaagctgctccacgctgggttcaaagggcgggtgacgggctggggcaaccggagggagacgtggaccaccagcgtggccgaggtgcagcccagcgtcctccaggtggtcaacctgcctctcgtggagcggcccgtgtgcaaagcctccacccggattcgcatcaccgacaacatgttctgtgccggttacaagcctggtgaaggcaaacgaggggacgcttgtgagggcgacagcgggggacccttcgtcatgaagagcccctataacaaccgctggtatcaaatgggcatcgtctcatggggtgaaggctgtgacagggatggaaaatatggcttctacacacacgtcttccgcctgaagaagtggattcagaaagttatcgaccgtctgggttcttaatag

SEQ ID NO: 4 - protein, 314 aa, Bovine engineered prethrombin-2
MAIEGRTSEDHFQPFFNEKTFGAGEADCGLRPLFEKKQVQDQTEKELFESYLVPRIVEGQDAEVGLSPWQVMLFRKSPQELLCGASLISDRWVLTAAHCLLYPPWDKNFTVDDLLVRIGKHSRTRYERKVEKISMLDKIYIHPRYNWKENLDRDIALLKLKRPIELSDYIHPVCLPDKQTAAKLLHAGFKGRVTGWGNRRETWTTSVAEVQPSVLQVVNLPLVERPVCKASTRIRITDNMFCAGYKPGEGKRGDACEGDSGGPFVMKSPYNNRWYQMGIVSWGEGCDRDGKYGFYTHVFRLKKWIQKVIDRLGS

SEQ ID NO: 5 - DNA, 1917 bp, Bovine engineered prothrombin
atggcccgcgtccgcggcccccggctgcctggctgcctggccctggctgccctgttcagcctcgtgcacagccagcatgtgttcctggcccatcagcaagcatcctcgctgctccagagggcccgccgtgccaacaagggcttcctggaggaggtgcggaagggcaacctggagcgagagtgcctggaggagccatgcagccgcgaggaggccttcgaggccctggagtctctcagtgccacggatgcgttctgggccaagtacacagcttgtgagtcagcgaaaaatcctcgagaaaagctcaatgaatgtctggaaggaaactgcgctgaaggtgtggggatgaactaccgagggaacgtgagcgtcacccggtcaggcatcgaatgccagctgtggagaagtcgctacccacataagccagaaatcaactctaccacccacccgggggctgacctgcgggagaatttttgccgcaacccggatggcagcattactgggccctggtgctacaccacatccccgactctgcggagagaagagtgcagcgtcccggtgtgcggccaggaccgagtcacagtggaggtgatcccccggtcaggaggctccactaccagtcagtcgcctttactggaaacatgcgtcccggaccgcggccgggagtaccgagggcggctggcggtgaccacaagcgggtcccgctgccttgcctggagcagcgaacaggccaaggccctgagcaaggaccaggacttcaacccggccgtgcccctggcggagaacttctgccgcaacccagacggggacgaggagggcgcctggtgctacgtggccgaccagcctggcgactttgagtattgtgacctgaactactgcgaggagccggtggatggagacctgggagacaggctgggtgaggacccggacccggacgcggccctggttccgcgtacgtctgaggaccatttccagcccttcttcaacgagaagacctttggcgccggggaggccgactgtggcctgcgacccctgttcgagaagaagcaggtgcaggaccaaacggagaaggagctcttcgagtcctacctggtaccgcgtatcgtggagggtcaggacgcggaggtaggcctctcgccctggcaggtgatgctctttcgtaagagtccccaggagctgctctgtggggccagcctcatcagtgaccgctgggtcctcacggctgcccactgtctcctgtacccgccttgggacaagaacttcactgtggatgacctgctggtgcgcatcggcaagcactcccgcaccaggtatgagcggaaggttgaaaagatctccatgctggacaaaatctacatccaccccaggtacaactggaaggagaatctggaccgggacatcgccctgctgaagctcaagaggcccatcgagttatccgactacatccaccccgtgtgcctgcccgacaagcagacagcagccaagctgctccacgctgggttcaaagggcgggtgacgggctggggcaaccggagggagacgtggaccaccagcgtggccgaggtgcagcccagcgtcctccaggtggtcaacctgcctctcgtggagcggcccgtgtgcaaggcctccacccggattcgcatcaccgacaacatgttctgtgccggttacaagcctggtgaaggcaaacgaggggacgcttgtgagggcgacagcgggggacccttcgtcatgaagagcccctataacaaccgctggtatcaaatgggcatcgtctcatggggtgaaggctgtgacagggatggaaaatatggcttctacacacacgtcttccgcctgaagaagtggatacagaaagtcattgatcggctgggaagcctggtgccccgcggcagccaccaccaccaccaccactgatga

SEQ ID NO: 6 - protein, 635 aa, Bovine engineered prothrombin
MARVRGPRLPGCLALAALFSLVHSQHVFLAHQQASSLLQRARRANKGFLEEVRKGNLERECLEEPCSREEAFEALESLSATDAFWAKYTACESAKNPREKLNECLEGNCAEGVGMNYRGNVSVTRSGIECQLWRSRYPHKPEINSTTHPGADLRENFCRNPDGSITGPWCYTTSPTLRREECSVPVCGQDRVTVEVIPRSGGSTTSQSPLLETCVPDRGREYRGRLAVTTSGSRCLAWSSEQAKALSKDQDFNPAVPLAENFCRNPDGDEEGAWCYVADQPGDFEYCDLNYCEEPVDGDLGDRLGEDPDPDAALVPRTSEDHFQPFFNEKTFGAGEADCGLRPLFEKKQVQDQTEKELFESYLVPRIVEGQDAEVGLSPWQVMLFRKSPQELLCGASLISDRWVLTAAHCLLYPPWDKNFTVDDLLVRIGKHSRTRYERKVEKISMLDKIYIHPRYNWKENLDRDIALLKLKRPIELSDYIHPVCLPDKQTAAKLLHAGFKGRVTGWGNRRETWTTSVAEVQPSVLQVVNLPLVERPVCKASTRIRITDNMFCAGYKPGEGKRGDACEGDSGGPFVMKSPYNNRWYQMGIVSWGEGCDRDGKYGFYTHVFRLKKWIQKVIDRLGSLVPRHHHHHH

SEQ ID NO: 7 - DNA, 1098 bp, Bovine engineered prethrombin-2
atggcccgcgtccgcggcccccggctgcctggctgcctggccctggctgccctgttcagcctcgtgcacagccagcatgtgttcctggcccatcagcaagcatcctcgctgctccagagggcccgccgtgcgacgtctgaggaccatttccagcccttcttcaacgagaagacctttggcgccggggaggccgactgtggcctgcgacccctgttcgagaagaagcaggtgcaggaccaaacggagaaggagctcttcgaatcctacctggtaccgcgtatcgtggagggtcaggacgcggaggtaggcctctcgccctggcaggtgatgctctttcgtaagagtccccaggagctgctctgtggggccagcctcatcagtgaccgctgggtcctcacggctgcccactgtctcctgtacccgccttgggacaagaacttcactgtggatgacctgctggtgcgcatcggcaagcactcccgcaccaggtatgagcggaaggttgaaaagatctccatgctggacaaaatctacatccaccccaggtacaactggaaggagaatctggaccgggacatcgccctgttgaagctcaagaggcccatcgagttatccgactacatccaccccgtgtgcctgcccgacaagcagacagcagccaagctgctccacgctgggttcaaagggcgggtgacgggctggggcaaccggagggagacgtggaccaccagcgtggccgaggtgcagcccagcgtcctccaggtggtcaacctgcctctcgtggagcggcccgtgtgcaaagcctccacccggattcgcatcaccgacaacatgttctgtgccggttacaagcctggtgaaggcaaacgaggggacgcttgtgagggcgacagcgggggacccttcgtcatgaagagcccctataacaaccgctggtatcaaatgggcatcgtctcatggggtgaaggctgtgacagggatggaaaatatggcttctacacacacgtcttccgcctgaagaagtggattcagaaagttatcgaccgtctgggttctctggtgccccgcggcagccaccaccaccaccaccactaatag

SEQ ID NO: 8 - protein, 362 aa, Bovine engineered prethrombin-2
MARVRGPRLPGCLALAALFSLVHSQHVFLAHQQASSLLQRARRATSEDHFQPFFNEKTFGAGEADCGLRPLFEKKQVQDQTEKELFESYLVPRIVEGQDAEVGLSPWQVMLFRKSPQELLCGASLISDRWVLTAAHCLLYPPWDKNFTVDDLLVRIGKHSRTRYERKVEKISMLDKIYIHPRYNWKENLDRDIALLKLKRPIELSDYIHPVCLPDKQTAAKLLHAGFKGRVTGWGNRRETWTTSVAEVQPSVLQVVNLPLVERPVCKASTRIRITDNMFCAGYKPGEGKRGDACEGDSGGPFVMKSPYNNRWYQMGIVSWGEGCDRDGKYGFYTHVFRLKKWIQKVIDRLGSLVPRHHHHHH